(19) United States
(12) Patent Application Publication
(10) Pub. No.: US 2007/0038015 A1
(43) Pub. Date: Feb. 15, 2007

(54) RECHARGEABLE IONIC-MAGNETIC BRACELET SYSTEM

(75) Inventor: David W. QUAIL, Woodstock, GA (US)

Correspondence Address: GARDNER GROFF SANTOS & GREENWALD, P.C., 2018 POWERS FERRY ROAD, SUITE 800, ATLANTA, GA, 30339, US

(73) Assignee: David W. QUAIL, Woodstock, GA (US)

(21) Appl. No.: 11/463,712

(22) Filed: Aug. 10, 2006

Related U.S. Application Data
(60) Provisional application No. 60/708,405, filed on Aug. 15, 2005.

(51) Int. Cl. A61B 17/52

(52) U.S. Cl. 600/9

(57) ABSTRACT

A rechargeable magnetic ionic bracelet system includes an ionic-magnetic bracelet and a recharger. The bracelet has an arm band portion and at least two terminals. The recharger can have a powerful magnet. The bracelet can be recharged by placing the terminals in contact with the magnetic poles of the recharger.

### CROSS-REFERENCE TO RELATED APPLICATION

The benefit of the filing date of U.S. Provisional Patent Application Ser. No. 60/708,405, filed Aug. 15, 2005, entitled RECHARGEABLE IONIC-MAGNETIC BRACELET, is hereby claimed and the specification thereof incorporated herein in its entirety by this reference.

### BACKGROUND OF THE INVENTION

Many people (e.g., athletes like golfers) have purchased and worn ionic/ionized bracelets for the claims that they provide health or performance results, such as decreased pain. Other claims of body balance, accuracy, speed, focus, and better overall performances have also been made.

One proposed theory of function of these bracelets is based on the Chinese theories of energy flow (chi) and balancing a flow of ions through the body using electro polarization.

Regardless of whether any of these health or performance results are true, any magnetization/ionization of the bracelets will discharge over time. Most sellers and/or wearers of these bracelets say that they need to be replaced about every 12 months to four years due to the ionization discharging. The present invention addresses this deficiency and others in the manner described below.

### SUMMARY

The present invention relates to a system comprising a rechargeable ionic-magnetic bracelet and a charger for recharging the bracelet. In an exemplary embodiment of the invention, the rechargeable ionic-magnetic bracelet can comprise an arm band portion, at least two bracelet terminals effective for recharging and holding an ionic charge for a period of time, and a charger for recharging the bracelet terminals. The bracelet terminals can be, for example, balls or spheres. The spherical terminals can be disposed at opposing ends of the arm band portion.

The charger can comprise an effective magnet that can be placed into contact with the bracelet terminals during charging. In an exemplary embodiment, the charger comprises an elongated permanent magnet, and each bracelet terminal can be placed into contact with one end (pole) of the magnet during charging. In the exemplary embodiment, resilience of the arm band portion of the bracelet can help hold the terminals against the ends of the charger magnet. Nevertheless, in other embodiments any other suitable charging arrangement can be provided. In an exemplary embodiment, the charger magnet can have, for example, the following properties:
(1) Nd—Fe—B, type N35 with zincification surface treatment,
(2) residual induction Br (KGs) of about 11.7-about 12.3,
(3) coercive force bHe(KOe) of about 10.7-about 12.0,
(4) intrinsic coercive force iGe (KOe) of about ≧12,
(5) squareness (Hk/kHe) of about ≧0.85,
(6) maximum energy product (BH) max. (MGOe) of about 33-about 36,
(7) maximum operating temperature of about 80° C.,
(8) density (g/cm) of about ≧7.40, and
(9) magnetic force of more than about 4500 gauss on each pole.

An embodiment of the invention is described in further detail below. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. Like numbers represent the same elements throughout the figures.

FIG. 1 is a side elevational view of an ionic-magnetic bracelet in accordance with an exemplary embodiment of the invention.

FIG. 2 is a top or edge view of the ionic-magnetic bracelet shown in FIG. 1.

FIG. 3 is a side elevational view of a charger for the ionic-magnetic bracelet shown in FIGS. 1-2 in accordance with an exemplary embodiment of the invention.

FIG. 4 is an end view of the charger shown in FIG. 3.

FIG. 5 is sectional view taken on line 5-5 of FIG. 4.

FIG. 6 is a side elevational view of the bracelet shown in FIGS. 1-2 and charger shown in FIGS. 3-5, with the bracelet in the charging position.

### DETAILED DESCRIPTION OF EXEMPLARY EMBODIMENT(S)

Before the present articles, devices, compositions, and/or methods are disclosed and described, it is to be understood that the aspects described below are not limited to specific synthetic methods or specific embodiments, as such may, of course, vary. It is also to be understood that the terminology used herein is for the purpose of describing particular aspects of one or more exemplary embodiments only and is not intended to be limiting of the scope of the invention.

In this specification and in the claims which follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “an alloy” includes mixtures of alloys, reference to “a metal” includes mixtures of two or more such metals, and the like.

“Optional” or “optionally” means that the subsequently described element, event or circumstance is present or may occur in some embodiments of the invention and not in others, and that the description includes instances or embodiments where the element is present or event or circumstance occurs and instances where it is not present or does not occur. For example, the phrase “optionally additional components” means that the additional components may be included in some embodiments and not in others, and that the description includes within its scope both a device with additional components and a device where there are no additional components.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

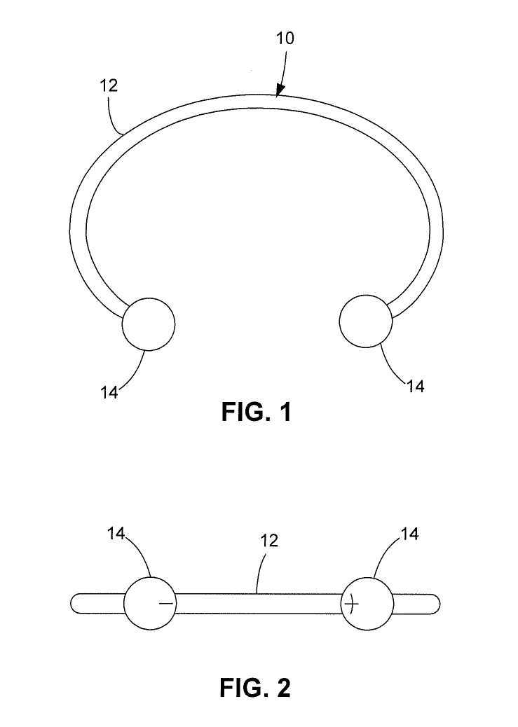

As illustrated in FIGS. 1-2, an ionic-magnetic bracelet 10 of the present invention for wearing on a person's wrist or other appendage combines magnetic therapy with ions. The bracelet 10 is also rechargeable, as described in further detail below. The bracelet 10 is assembled from an arm band 12 and bracelet terminals 14 using techniques known in the art. The bracelet 10 is initially ionized when constructed using ionization techniques generally known in the art (e.g., ionization can be done in a vacuum chamber using methods such as those used for ionizing other structures, for example, circuit boards).

The bracelet terminals 14 are preferably spherical or substantially spherical in shape (i.e., ball-shaped) and made of a magnetizable base metal or alloy. In the exemplary embodiment, they are steel, coated or plated with a layer of 24k gold about 6 microns in thickness. In other embodiments they can comprise other suitable materials, such as titanium-plated gold. For example, the gold-titanium can be plated onto the other metal in a vacuum. It is believed that the gold-titanium plating will seal the bracelet terminals 14 cosmetically as well as help generate more ions. Standard techniques for producing plated or coated metal objects can be used to fabricate the bracelet terminals 14. One of skill in the art can determine various ways of manufacturing the bracelet terminals 14. As described below, the bracelet terminals 14 store a magnetic charge and release ions over time. As best seen in FIG. 2, bracelet terminals 14 can have positive (“+”) and negative (“−”) symbols (e.g., by embossing or other suitable method) to indicate polarities.

The arm band 12 supports the bracelet terminals 14 and allows bracelet 10 to be conveniently worn on an appendage of a wearer, such as a wrist or arm. The arm band 12 separates the bracelet terminals 14 and does not itself cause the bracelet terminals 14 to discharge. In the exemplary embodiment, the arm band 12 is generally C-shaped, with the bracelet terminals 14 disposed on the opposing ends of the arm band 12, thus leaving a gap in between them. The arm band 12 can comprise a metal. In the exemplary embodiment, the metal is stainless steel, fashioned in the manner of a multi-stranded wire rope.

It is preferred that the arm band 12 be sized or sizable to fit the wearer. It is also preferred that the arm band 12 be flexible and have some resilience so that a user can spread or expand the ends apart wide enough to insert his or her wrist, then allow the arm band 12 to resiliently return or spring back to its relaxed state around the wrist.

When worn, the magnetic field emanating from bracelet 10 is believed to act like a powerful booster for the ions through electro-polarization, and this electric charge is believed to create a passive-energy resonator, providing the wearer with possible therapeutic effects. It is believed that ions “jump” between the bracelet terminals 14. The body of the wearer is intended to be the path through which the ions travel as well as any magnetic field.

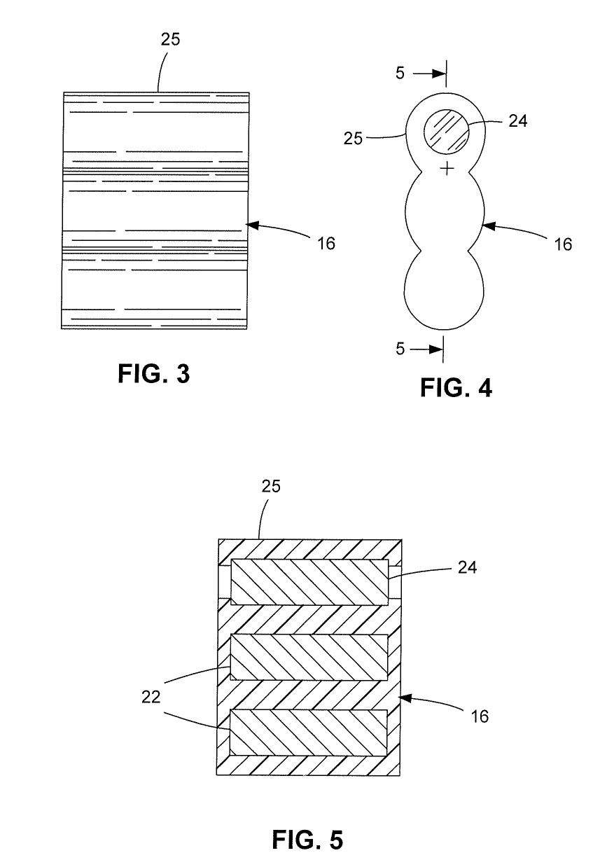

As illustrated in FIGS. 3-5, a charger 16 can be used to recharge bracelet 10. In the exemplary embodiment of the invention, the charger 16 comprises a powerful permanent magnet 24 embedded in a case 25 with its ends (i.e., positive and negative poles) exposed through openings in the case 25. In the exemplary embodiment, case 25 is made of a hard and durable rubber-like material, but in other embodiments it can be made of other suitable materials. Positive (“+”) and negative (“−”) symbols can be printed or otherwise provided on case 25 to indicate the polarities of the exposed ends (poles) of magnet 24. Although in the exemplary embodiment of the invention magnet 24 is a permanent magnet, in other embodiments it can comprise an electromagnet or other suitable device.

In the exemplary embodiment, the magnet 24 can comprise a conventional Nd—Fe—B magnet. The surface of magnet 24 can have zincification. A suitable magnet type is N35. A magnet 24 having a residual induction Br of between about 11.7 and about 12.3 KGs is suitable. Further, a magnet 24 having a coercive force bHe of between about 10.7 and about 12.0 Koe and intrinsic coercive force iGe of about ≧12 KOe is suitable. The squareness (Hk/kHe) of this exemplary magnet is about ≧0.85. The maximum energy product of the exemplary magnet 24 (BH max.) is between about 33 to about 36 MGOe. The maximum operating temperature of the exemplary magnet 24 is about 80° C. The density of the exemplary magnet is about ≧7.40 g/cm. The magnetic force is more than about 4500 gauss on each pole. The exemplary magnet 24 is cylindrical in shape, with a length of about 1⅞ inches and a radius of about 9/16 inches.

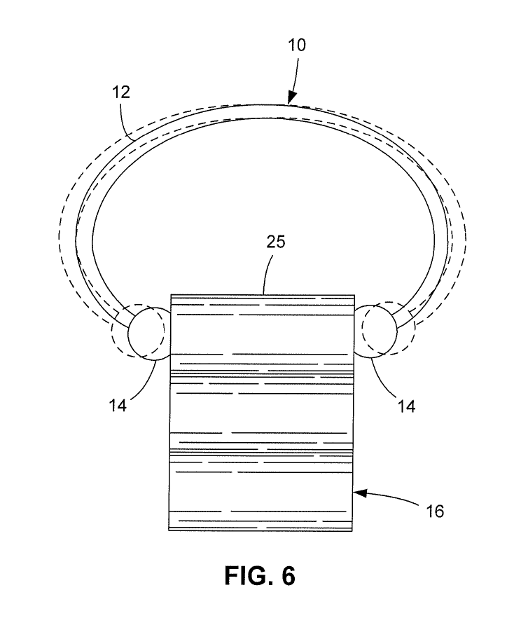

To charge bracelet 10, a user places bracelet terminals 14 in contact with the poles of magnet 24 of charger 16, as illustrated in FIG. 6. As described above, charger 16 and bracelet terminals 14 are marked with pole indicia (“+” and “−”) to indicate their polarities. A user can accordingly use the indicia to line up the positive and negative terminals 14 of bracelet 10 with the corresponding positive and negative poles of magnet 24 of charger 16. As arm band 12 has some degree of resiliency, the user can spread bracelet terminals 14 apart slightly as indicated in dashed line, slip them over charger 16 as shown, and then release them, allowing the arm band 12 to resiliently return or spring back to its relaxed state as shown, with bracelet terminals 14 held in place at least in part by their engagement in the circular openings in case 25.

In the charging (or, it can equivalently be said, recharging) position shown in FIG. 6, the charger 16 can place ions on the terminals 14 of bracelet 10. Other such bracelets known in the art discharge when touching other metals and eventually have to be replaced. The charger 16 of the present invention enables one to recharge bracelet 10 to the magnetization with which it was originally manufactured. Recharging in this manner replenishes the ions, providing a continuous flow of ions and a magnetic charge, ensuring the bracelet 10 is always at full strength. This allows the consumer to effectively recharge their rechargeable bracelet. It is believed that a magnet 24 with at least the above-described magnetic force (about 4500 gauss each pole) will transfer a magnetic field to the terminals 14 of the bracelet 10 to cause a sufficient transference of ions to allow the bracelet 10 to recharge to its original state.

In addition to magnet 24, charger 16 contains weights 22 (FIG. 5) that help it hang in a convenient orientation between the bracelet terminals 14 when in the charging position.

The charger 16 and the bracelet 10 can be sold or otherwise provided together as a system or kit.

The bracelet 10 can be placed on the wrist and worn like other bracelets of its type. A person can perform the above-described method for recharging bracelet 10 at any time, such as daily or at other regular intervals. For example, the user can perform the recharging method when he or she feels the effects of the bracelet wearing off. As another example, the user can perform the recharging method while sleeping or showering. The recharging method can be performed, for example, overnight (e.g., 6-8 hours), or any period of time sufficient to transfer ions onto the terminals 14.

It will be apparent to those skilled in the art that various modifications and variations can be made to this invention without departing from the spirit or scope of the invention. Thus, it is intended that the present invention cover all modifications and variations of this invention that come within the scope of one or more claims and their equivalents. With regard to the claims, no claim is intended to invoke the sixth paragraph of 35 U.S.C. Section 112 unless it includes the term “means for” followed by a participle.

What is claimed is:

1. A rechargeable ionic-magnetic bracelet system, comprising:
an arm band portion that does not discharge ions;
at least two terminals connected to the arm band portion for recharging and holding an ionic charge for a period of time; and
a magnetic charger for recharging the terminals.

2. The bracelet system of claim 1, wherein the terminals are connected to opposing ends of the arm band portion and separated by a gap.

3. The bracelet system of claim 1, wherein the arm band portion is metal.

4. The bracelet system of claim 1, wherein the arm band portion is resilient.

5. The bracelet system of claim 1, wherein the terminals are substantially spherical.

6. The bracelet system of claim 1, wherein the terminals comprise a magnetizable metal.

7. The bracelet system of claim 1, wherein the terminals comprise gold-plated steel.

8. The bracelet system of claim 1, wherein the terminals comprise gold-titanium plated steel.

9. The bracelet system of claim 1, wherein the magnetic charger comprises:
a case; and
a permanent magnet disposed at lest partly in the case, with poles of the magnet exposed to an exterior of the case to facilitate contact with the terminals.

10. The bracelet system of claim 9, wherein the case is made of a molded material, and the magnet is embedded in the case with positive and negative poles of the magnet exposed through openings in the case.

11. The bracelet system of claim 10, wherein the openings have a depth to provide engagements for the terminals.

12. The bracelet system of claim 10, wherein the case is made of an elastomeric material.

13. The bracelet system of claim 9, wherein the magnet comprises Nd—Fe—B.

14. The bracelet system of claim 9, wherein the magnet has a magnetic strength of equal to or greater than about 4500 gauss on each pole.

15. The bracelet system of claim 9, wherein the magnet has a residual induction Br (KGs) of about 11.7 to about 12.3.

16. The bracelet system of claim 9, wherein the magnet type is N35.

17. The bracelet system of claim 9, wherein the magnet has a coercive force bHe(KOe) about 10.7 to about 12.0.

18. The bracelet system of claim 9, wherein the magnet has an intrinsic coercive force iGe (KOe) of about ≧12.

19. The bracelet system of claim 9, wherein the magnet has a squareness (Hk/kHe) of about ≧0.85.

20. The bracelet system of claim 9, wherein the magnet has a maximum energy product of the magnet BH max (MGOe) of about 33 to about 36.

21. The bracelet system of claim 9, wherein the magnet has a density (g/cm) of about ≧7.40.

22. The bracelet system of claim 9, wherein the magnet comprises:
(a) Nd—Fe—B, type N35 with zincification surface treatment, and wherein the properties include
(i) a residual induction Br (KGs) of about 11.7-about 12.3,
(ii) a coercive force bHe(KOe) of about 10.7-about 12.0,
(iii) an intrinsic coercive force iGe (KOe) of about ≧12,
(iv) a squareness (Hk/kHe) of about ≧0.85,
(v) a maximum energy product (BH) max. (MGOe) of about 33-about 36,
(vi) a maximum operating temperature of about 80° C.,
(vii) a density (g/cm) of about ≧7.40, and
(viii) a magnetic force of more than about 4500 gauss on each pole.

23. A method for charging an ionic-magnetic bracelet, the bracelet comprising at least two terminals capable of holding an ionic charge for a period of time and an arm band portion that does not discharge ions, the method comprising:
placing the terminals of the bracelet into contact with magnetic poles of a charger.

24. The method claimed in claim 23, wherein the bracelet comprises terminals connected to opposing ends of a substantially resilient arm band and separated by a gap, and the placing step comprises:
spreading the terminals to widen the gap;
placing the terminals adjacent the magnetic poles of the charger; and
releasing the terminals to allow the terminals to resiliently retract into contact with the magnetic poles of the charger.

25. The method claimed in claim 24, wherein the charger has a case with openings through which the magnetic poles are exposed, and the placing step comprises placing the terminals into the openings.

26. The method claimed in claim 25, wherein the openings are annular, the terminals are substantially spherical, and the terminals engage the annular openings.
